(19) United States
(12) Patent Application Publication
(10) Pub. No.: US 2023/0047711 A1
(43) Pub. Date: Feb. 16, 2023

(54) FLOWABLE COLLAGEN COLLOID AND METHOD OF FORMING

(71) Applicant: Sasan Najibi, Calabasas, CA (US)

(72) Inventor: Sasan Najibi, Calabasas, CA (US)

(21) Appl. No.: 17/402,473

(22) Filed: Aug. 13, 2021

(51) Int. Cl. A61L 26/00

(52) U.S. Cl. CPC A61L 26/008; A61L 26/0042; A61L 26/0033; A61L 2300/418; A61L 2400/06

(57) ABSTRACT

A hemostatic colloid and method of forming a hemostatic colloid for dispensing into a wound site to control bleeding is provided. The hemostatic colloid is comprised of collagen in microfibril, crystalline, powder or granular form and is mixed with a liquid thrombin solution to form a flowable collagen/thrombin hemostatic colloid.

### BACKGROUND OF THE INVENTION

### 1. Field of the Invention

The present invention relates to methods of forming and using a hemostatic materials, and more specifically to collagen based hemostatic materials and method of forming a hemostatic colloid used to control bleeding and oozing from a variety of wounds.

### 2. Background and Related Art

Surgical procedures and injuries are often associated with blood loss. Conventional approaches for dealing with blood loss, such as manual pressure, cauterization, or suture ligation can be time consuming and may not be effective in controlling bleeding/oozing.

A number of topical hemostatic agents have been developed to control bleeding resulting from surgical procedures and injury. Some hemostatic agents, such as collagen-based powders, sponges, and cloths, are of a particulate nature. Such particulate hemostatic agents provide a lattice for natural thrombus formation, but are unable to enhance this process in coagulopathic patients. Pharmacologically-active agents, such as thrombin, can be used in combination with a particulate carrier, for example, as in a gel-foam sponge or powder soaked in thrombin, and/or gelatin. Thrombin has been used to control bleeding on diffusely bleeding tissue surfaces, but the lack of a framework onto which the clot can adhere has limited its utility. The autologous and allogenic fibrin glues can cause clot formation, but do not adhere well to wet tissue and have little impact on actively bleeding wounds.

The use of collagen to form a hemostatic collagen paste is disclosed in U.S. Pat. No. 4,891,359. The hemostatic collagen paste composition comprises a mixture by weight of the total composition of 2 to 30% of crosslinked collagen powder of 10 to 100 mesh particle size and 30 to 98% water or an aqueous saline solution. In a particular embodiment, the hemostasis effect of the collagen paste composition are further enhanced by adding a hemostatic enhancing effective amount of thrombin to the paste composition. A sterile thrombin solution is prepared by mixing together dry thrombin with isotonic saline and this solution is mixed with collagen powder to make a paste or dough. For example, 1 gram of collagen powder is mixed with 10 ml of thrombin solution containing 10,000 units of thrombin. The paste resulting from this is a 9% collagen paste (1 gram collagen in a total of 11 grams of paste) containing 10,000 units of thrombin distributed throughout the paste. The ratio of thrombin to collagen is 10,000 units thrombin per gram of collagen. The ratio of thrombin to collagen can be much less such as 100 units of thrombin per gram of collagen or even much higher such as 20,000 units per gram of collagen. According to U.S. Pat. No. 4,891,359, however, “[i]t is critical that the collagen materials of the invention be crosslinked in order to form a usable paste composition. Small uncrosslinked collagen fibers, such as for example those contained in AVITENE brand collagen fibers, are not stable in the form of a wet paste because uncrosslinked collagen deteriorates and gels upon wetting. The crosslinking of the collagen fibers also contributes to its integrity in a paste form which does not become tacky or gel-like when handled with wet surgical gloves and can be readily packed into a wound without sticking to the gloves. Similarly, U.S. patent application Ser. No. 15/666,222 discloses a flowable hemostatic composition comprising crosslinkable collagen molecules. Also known as coagulation factor II, thrombin is a serine protease that plays a physiological role in regulating hemostasis and maintaining blood coagulation. Once converted from prothrombin, thrombin converts fibrinogen to fibrin, which, in combination with platelets from the blood, forms a clot.

Medical thrombin is typically a topical bovine thrombin indicated to aid hemostasis whenever oozing blood and minor bleeding from capillaries and small venules is accessible and to control bleeding where standard surgical techniques (such as suture, ligature, or cautery) is ineffective or impractical. In various types of surgeries, solutions of thrombin have been used in conjunction with an absorbable gelatin sponge, USP for hemostasis.

For routine use of thrombin, it is reconstituted with sterile isotonic saline. Where bleeding is profuse, as from abraded surfaces of the liver or spleen, higher concentrations of thrombin may be required. Thrombin is routinely used in plastic surgery, dental extractions, skin grafting, etc. Thrombin has also been used in a dry form on oozing surfaces.

Microfibrillar collagen products, like AVITENE (Davol, Inc.) microfibrillar collagen hemostat, are made by purifying bovine collagen and processing it into microcrystals. Indications for application of AVITENE microfibrillar collagen hemostat are to apply it in its dry form to a wound site where bleeding is occurring. This procedure is difficult and often results in the AVITENE microfibrillar collagen hemostat adhering the gloves of the surgeon rather than to the wound site and clumping together.

The aforementioned patents disclose use of crosslinked or crosslinkable collagen molecules to form hemostatic compositions and expressly teach away from the use of noncrosslinked collagen particles, such as AVITENE brand microfibrillar collagen hemostat, which according to U.S. Pat. No. 4,891,359 are not stable in the form of a wet paste because uncrosslinked collagen deteriorates and gels upon wetting. Such uncrosslinked collagen, however, when used to form a colloidal hemostat as shown and described in the invention set forth herein has significantly and surprisingly better hemostatic properties than hemostats of the prior art. Accordingly, a hemostatic colloid and method of forming a hemostatic colloid into a stable gelatinous or gel-like form and method of applying the hemostatic colloid in such a form to control bleeding from a variety of wounds is desirable.

### SUMMARY OF THE INVENTION

The present invention relates to a method of forming a hemostatic colloid for use in controlling bleeding from a variety of wounds and. In particular, the present invention relates to the formation of a hemostatic colloid from a solid hemostatic material made from uncrosslinked collagen in a solid form mixed with a thrombin/saline solution. The resulting flowable hemostatic collagen colloid is suitable for controlling active bleeding and oozing.

A method of preparing hemostatic colloid comprises admixing a plurality of collagen particles with a hemostatic agent in the form of a liquid thrombin solution and mixing the plurality of collagen particles with the liquid thrombin solution until a flowable hemostatic colloid is formed. The collagen particles have a bulk density sufficient to form a suspension in the liquid thrombin solution and wherein the flowable hemostatic colloid has a collagen concentration in the range of approximately 10%-20% weight/volume.

In one embodiment, the collagen particles comprise uncrosslinked collagen microfibrils. The collagen particles may have a bulk density in a range of approximately 1.5 lbs/ft³ to about 3.5 lbs/ft³. The collagen particles may also be in the form of a powder or in a crystalline or granular form.

In another embodiment, the flowable hemostatic colloid has a viscosity of less than 3300 centipoise. For example the flowable hemostatic colloid may have an absolute viscosity of between approximately 1000 centipoise and approximately 3000 centipoise.

In another embodiment, the flowable hemostatic colloid has a pH in a range of approximately 4.0 to 6.0. For example, the flowable hemostatic colloid may have a pH of 4.5-5.5 or a pH of approximately 5.0+/−0.1.

In yet another embodiment, the pH of the flowable hemostatic colloid is increased by adding sodium bicarbonate to either the collagen particles or the liquid thrombin solution prior to mixing. For example, by mixing approximately 1 gram of collagen particles with approximately 8 mL liquid thrombin solution and approximately 2 mL sodium bicarbonate, the flowable hemostatic colloid has a pH of 7.1. By mixing approximately 1 gram collagen particles with approximately 9 mL liquid thrombin solution and approximately 1 mL sodium bicarbonate to form the flowable hemostatic colloid has a pH of approximately 6.5.

In one embodiment, the liquid thrombin solution is formed by mixing thrombin with saline.

In another embodiment, the flowable hemostatic colloid comprises a mixture of approximately 1 gram of the collagen particles with approximately 10 mL of the liquid thrombin solution.

In still another embodiment, connecting a first syringe containing the liquid thrombin solution to a second syringe containing the collagen particles and air, a portion of the liquid thrombin solution can be dispensed from first syringe into the second syringe to wet the collagen particles. By agitating the first and second connected syringes by shaking, tipping, rolling and/or rotating the first and second syringes in a back and forth motion clumping of the collagen particles can be prevented. The remainder of the thrombin solution can then be dispensed from the first syringe into the second syringe to form a collagen/thrombin mixture. Passing the collagen/thrombin mixture back and for the between the first and second syringes is performed until the flowable hemostatic colloid is formed.

In another embodiment, a flowable hemostatic colloid according to the present invention is comprised of a plurality of uncrosslinked collagen particles homogenously mixed with a hemostatic agent in the form of a liquid thrombin solution. The collagen particles have a bulk density sufficient to form a suspension in the liquid thrombin solution. The flowable hemostatic colloid has a collagen concentration in the range of approximately 10%-20% weight/volume.

The collagen may be in the form of microfibrils, microcrystals, granules or a powder. The collagen is then combined with a thrombin solution, safe for injection into or use on a human body, to form a hemostatic colloid in a gelatinous or foam-like form, hereinafter referred to as a flowable hemostatic collagen colloid. For example, the liquid may comprise a solution of sterilized water or saline combined with thrombin. A foaming agent, such as albumin may also be added. In addition, the collagen colloid may be combined with other materials and/or substances such antibiotics for additional benefits. The collagen may be admixed with the thrombin liquid by various methods. For example, the flowable hemostatic collagen colloid may be formed by connecting two syringes (such as two 20 ML syringes) to a three-way-stopcock or a female-female connector in between, or a male-female syringe connected directly. One syringe contains the solid collagen material and the other syringe contains the liquid. The liquid from one syringe is forced through the connector into the syringe containing the solid hemostatic material. The liquid and hemostatic material is then passed back and forth from one syringe to the other until a uniform consistency is achieved (e.g., 30 seconds to 2 minutes). The mixing of the solid collagen material with the liquid forms a flowable collagen colloid in the form of a gel or liquid-foam that can be injected into a desired position with one of the syringes containing the flowable collagen colloid after mixing.

The flowable hemostatic collagen colloid may be used both outside and inside the body and can be absorbed by the human body. Because of the coagulation properties of the flowable hemostatic collagen colloid, hemostasis may be fast, e.g., in the order of a few seconds. Depending upon the nature of the wound and the treatment method employed, the flowable hemostatic collagen colloid can be fabricated in various forms for controlling the active bleeding from an artery or vein, or for controlling internal bleeding during laparoscopic procedures, surfaces of bone in orthopedic procedures, or bleeding from solid organs. The flowable hemostatic collagen colloid can be safely injected into or around tissue to achieve hemostasis. In addition, such a flowable hemostatic collagen colloid does not present a risk of obstruction or compression of pressure sensitive organs or tissues due to excessive swelling. Such a flowable hemostatic collagen colloid is specifically applicable in neuro/spine, laparoscopic, orthopedic, general, cardiac and vascular surgery.

The solid hemostatic collagen material may be provided in a kit containing two syringes with a first syringe containing the solid collagen hemostatic material in microfibrillar, microcrystal, powder or granular form and a second syringe containing the desired thrombin liquid. Using a three-way stopcock or female to female connector connected between the two syringes, or special male-female syringes, the solid hemostatic material can be mixed with the liquid to form a gel in the form of a flowable collagen colloid at the time when the hemostatic colloid is needed. Alternatively, the flowable hemostatic collagen colloid is provided in a pre-made flowable hemostatic collagen colloid form in a single syringe. In yet another embodiment, the flowable hemostatic collagen colloid is provided within a pressurized container containing a liquid foaming agent so that a foamed hemostatic material can be dispensed as needed. Thus, regardless of the form in which the hemostatic material is provided, it is easy to carry and store, is stable, has a relatively long shelf life, meets the requirements of surgery and daily use, can be applied for emergency hemostasis in the battle ground, causes no pain, conforms to wounds accurately, adheres well to wound sites, even when wet, has no known side effects, and exhibits high hemostasis efficacy-even in patients with a blood-coagulation defect or patients on blood-thinning drugs. The flowable hemostatic collagen colloid of the invention is simple to form, safe and easy to use, economical, can be utilized under any circumstances where hemostasis is needed and can be made economically in the industry.

These and other features and advantages of the present invention will be set forth or will become more fully apparent in the description that follows and in the appended claims. The features and advantages may be realized and obtained by means of the instruments and combinations particularly pointed out in the appended claims. Furthermore, the features and advantages of the invention may be learned by the practice of the invention or will be obvious from the description, as set forth hereinafter.

### BRIEF DESCRIPTION OF THE DRAWINGS

In order that the manner in which the above recited and other features and advantages of the present invention are obtained, a more particular description of the invention will be rendered by reference to specific embodiments thereof, which are illustrated in the appended drawings. Understanding that the drawings depict only typical embodiments of the present invention and are not, therefore, to be considered as limiting the scope of the invention, the present invention will be described and explained with additional specificity and detail through the use of the accompanying drawings in which:

FIG. 1 is an image of uncrosslinked microfibrillar collagen in accordance with the principles of the present invention.

FIG. 2 is a first device and method of forming the collagen-based hemostatic colloid in accordance with the principles of the present invention.

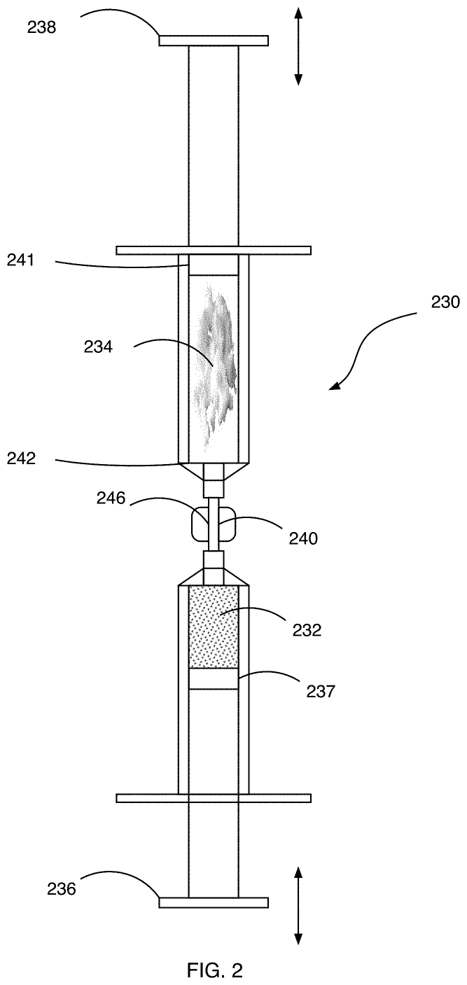

FIG. 3 is a device and method of dispensing the collagen-based hemostatic colloid in accordance with the principles of the present invention.

### DETAILED DESCRIPTION OF THE INVENTION

The present invention relates to a method of forming a hemostatic flowable collagen colloid that is bioabsorbable and can be formed on site for use in controlling bleeding from a variety of wounds. In particular, the present invention relates to a flowable hemostatic colloid made from collagen. The hemostatic flowable collagen colloid is suitable for controlling active bleeding and oozing from tissues.

1. Hemostasis

To better explain the flowable hemostatic collagen colloid of the invention, a non-binding description of hemostasis is provided herein. The term hemostasis may be used to refer to the mechanism (e.g., normal vasoconstriction, abnormal obstruction, coagulation, or surgical means) that stems bleeding after injury to the vasculature. Biological hemostasis depends on both cellular components and soluble plasma proteins. In particular, hemostasis by coagulation may be dependent upon a complex interaction of plasma coagulation and fibrinolytic proteins, platelets, and the blood vasculature. The hemostatic process may be conceptually separated into three stages: primary hemostasis, secondary hemostasis, and tertiary hemostasis.

Primary hemostasis may principally be characterized by the formation of a primary platelet plug. The plug may be formed as circulating platelets adhere and aggregate at sites of blood vessel injury. In areas of high shear rate (e.g., microvasculature) aggregation may be mediated by von Willebrand factor (vWf), which may bind to glycoprotein Ib-IX in the platelet membrane. In areas of low shear rate (e.g., arteries) fibrinogen mediates the binding of platelets to the subendothelium by attaching to a platelet receptor. Aggregation begins with platelets adhering to exposed subendothelium (Collagen). When platelets adhere to the vessel wall, they change shape and activate the collagen receptor on their surface to release alpha and dense granule constituents. Injury to the blood vessel wall is additionally followed by vasoconstriction. Vasoconstriction not only retards extravascular blood loss, but also slows local blood flow, enhancing the adherence of platelets to exposed subendothelial collagen surfaces and the activation of the coagulation process.

Formation of the plug may be followed by an aggregation response. Activation of platelets results in exposure of anionic phospholipids that serve as platforms for the assembly of blood coagulation enzyme complexes. Platelet aggregation involves the activation, recruitment, and binding of additional platelets to the adhered platelets. Aggregation is promoted by platelet agonists, such as thromboxane 2, PAF, ADP, and serotonin. Activated platelets synthesize and release thromboxane and platelet activating factor, which are potent platelet aggregating agonists and vasoconstrictors. Activation is enhanced by the generation of another platelet agonist, thrombin (Factor II), through the coagulation cascade. Platelet aggregation is mediated primarily by fibrinogen, which binds to glycoprotein IIb/IIIa on adjacent platelets. This aggregation leads to the formation of the primary platelet plug, and is stabilized by the formation of fibrin.

Secondary hemostasis may be characterized by fibrin formation through the coagulation cascade, which involves circulating coagulation factors, calcium, and platelets. The coagulation cascade involves three pathways: intrinsic; extrinsic; and common. The main pathway for initiation of coagulation is the extrinsic pathway, while the intrinsic pathway acts to amplify the coagulation cascade.

The extrinsic pathway may involve the tissue factor and factor VII complex, which activates factor X. The extrinsic pathway of blood coagulation is initiated when blood is exposed to tissue factor. Tissue factor, a transmembrane protein, is expressed by endothelial cells, subendothelial tissue (Collagen) and monocytes, with expression being upregulated by cytokines. Tissue factor binds activated factor VII (factor Vila) and the resulting complex activates factors X and IX. Factor X, in the presence of factor V, calcium, and platelet phospholipid, then activates prothrombin to thrombin. This pathway is rapidly inhibited by a lipoprotein-associated molecule referred to as tissue factor pathway inhibitor. However, the small amount of thrombin generated by this pathway activates factor XI of the intrinsic pathway, which amplifies the coagulation cascade.

Thrombin activates the intrinsic pathway by activation of factors XI and VIII. In the intrinsic pathway activated factor IX (factor IXa) combines with factor VIIIa to provide a second means to activate factor X. The intrinsic pathway involves high-molecular weight kininogen, prekallikrein, and factors XII, XI, IX and VIII. Factor VIII acts as a cofactor (with calcium and platelet phospholipid) for the factor IX-mediated activation of factor X. Activated factor IX, together with activated factor VIII, calcium, and phospholipid, referred to as tenase complex, amplify the activation of factor X, generating large amounts of thrombin.

The extrinsic and intrinsic pathways converge at the activation of factor X. The common pathway involves the factor X-mediated generation of thrombin from prothrombin (facilitated by factor V, calcium and platelet phospholipid), with the production of fibrin from fibrinogen. Factor Xa complexes with factor Va and prothrombin to form prothrombinase, which cleaves prothrombin to generate thrombin, the key enzyme in hemostasis. In the final step of the coagulation cascade, thrombin cleaves fibrinogen to generate fibrin monomers, which then polymerize. This polymer is covalently cross-linked by factor XIIIa (itself generated from factor XIII by thrombin) to form a chemically stable clot. Thrombin also feeds back to activate cofactors V and VIII, thereby further amplifying the coagulation system.

Tertiary hemostasis is characterized by the formation of plasmin, which is the main enzyme responsible for fibrinolysis. At the same time as the coagulation cascade is activated, tissue plasminogen activator is released from endothelial cells. Tissue plasminogen activator binds to plasminogen within the clot, converting it into plasmin. Plasmin lyses both fibrinogen and fibrin in the clot, releasing fibrin and fibrinogen degradation products.

Finally, fibrin is digested by the fibrinolytic system, the major components of which are plasminogen and tissue-type plasminogen activator (tPA). Both of these proteins are incorporated into polymerizing fibrin, where they interact to generate plasmin, which, in turn, acts on fibrin to dissolve the preformed clot.

The fibrinolytic system is, in turn, regulated by three serine proteinase inhibitors, namely, antiplasmin, plasminogen activator inhibitor-1 (PAI-1), and plasminogen activator inhibitor-2 (PAI-2). Plasma D-dimers are generated when the endogenous fibrinolytic system degrades fibrin. They consist of two identical subunits derived from two fibrin molecules. Unlike fibrinogen degradation products, which are derived from fibrinogen and fibrin, D-dimers are a specific cross-linked fibrin derivative

The process of fibrin deposition is limited by mechanisms of the natural anticoagulant system. The maintenance of adequate blood flow and the regulation of cell surface activity limit the local accumulation of activated blood coagulation enzymes and complexes. Antithrombin (AT) is a plasma protein member of the serpin (serine protease inhibitor) family that inhibits the activities of all of the activated coagulation enzymes. The inhibitory effect of AT is increased several thousand-fold by binding to heparin. Protein C is a vitamin K-dependent protein that proteolyses factor Va and factor VIIIa to inactive fragments. Protein C binds to an endothelial cell protein C receptor (EPCR) and is activated by thrombin bound to thrombomodulin, another endothelial cell membrane-based protein, in a reaction that is modulated by a cofactor, protein S. Tissue factor pathway inhibitor is a lipoprotein-associated plasma protein that forms a quaternary complex with tissue factor, factor Vila, and factor X, thereby inhibiting the extrinsic coagulation pathway.

2. Hemostatic Mechanism

The following is a description of the ways in which the hemostatic colloid of the invention may contribute to achieving hemostasis:

a) Hemostasis Through Physical Path

When the flowable hemostatic collagen colloid contacts blood, the hemostatic material may stimulate a blood clotting pathways. For example, the hemostatic collagen material, which has absorbed the water or saline and thrombin solution and thereby changed from a solid to a gelatinous substance, can slow the flow of the blood. The flowable hemostatic collagen colloid may cover the wound surfaces and further expand after as it absorbs additional fluid. As the flowable hemostatic collagen colloid contacts fluid in the blood, some part of the hemostatic material may form a viscous body and clog the end of capillary blood vessels.

b) Hemostasis Through Chemical Path

The term “Hemostasis through chemical path” means that when the flowable hemostatic collagen colloid of the invention contacts platelets, absorption and coagulation may occur at an increased rate.

c) Hemostasis Through Physiology Path

The term “Hemostasis through physiology path” means that the hemostatic flowable collagen colloid of the invention can activate the coagulation factors in the human body and boost the formation of thrombin so as to generate hemostasis efficacy. The coagulation factor may be the key factor to activate the endogenous coagulation system as well as the external coagulation system. It is already known that some coagulation factors may bring positive electricity; therefore, they could be generally activated by a substance with negative electricity. Because the hemostatic flowable collagen colloid may be water-soluble, it can generate large quantities of negative electricity to activate the coagulation factors.

3. Hemostatic Flowable Collagen Colloid

The various embodiments provide compositions and materials that react with the hemostatic system to treat or prevent bleeding. The compositions and materials of the embodiments may result in coagulation of blood. In particular, the compositions and materials are combined to form a hemostatic flowable collagen colloid. A flowable collagen colloid is a substance that forms a colloid in the presence of water or thrombin. In the present invention, the hemostatic colloid is formed by combining a solid hemostatic, such as collagen particle material with a liquid, such as water, saline or thrombin.

Effective delivery of hemostatic agents to wounds is desirable in the treatment of injuries characterized by bleeding, as well as in surgical procedures where the control of bleeding can become problematic (e.g., surgical procedures involving large surface areas, heavy arterial or venous bleeding, oozing wounds, organ laceration/resection, etc.). The compositions and materials of invention can possess a number of advantages in delivery of hemostatic agents to wounds, including, but not limited to, ease of application and removal, bioabsorption potential, antigenicity, and tissue reactivity.

Depending upon the nature of the wound and the treatment method employed, the embodiments of the hemostatic flowable collagen colloid can be fabricated in various forms. For example, a colloid, when applied to a wound can control active bleeding from an artery or vein, or control internal bleeding during laparoscopic procedures. In neurosurgery, where oozing brain wounds are commonly encountered, the flowable collagen colloid can be applied where other solid forms of hemostatic material may be difficult to apply. Despite differences in delivery and handling characteristics of the various forms, the hemostatic colloid may be effective in deploying hemostatic agents to an affected site and rapidly initiating hemostatic plug formation through platelet adhesion, platelet activation, and/or blood coagulation.

The flowable hemostatic colloid is formed by combining collagen in solid form with a thrombin based liquid solution, such as thrombin mixed with sterile water or saline. The flowable hemostatic colloid is formed from collagen that is combined with the liquid and mixed for a period of time until the solid hemostatic material absorbs the liquid and forms a flowable colloid. The solid hemostatic material can be provided in the form of microfibrils, microcrystals or granules of a pre-selected size or a powder. The resulting hemostatic colloid, while being provided with sufficient liquid to be flowable, as through a syringe, has the ability to further absorb other liquid, such as blood when applied to a wound to activate blood-coagulation.

A method of preparing a flowable hemostatic colloid according to the invention comprises admixing a plurality of collagen particles with a hemostatic agent in the form of a liquid thrombin solution. The plurality of collagen particles are mixed with the liquid thrombin solution until a flowable hemostatic colloid is formed. The collagen particles have a bulk density sufficient to form a suspension in the liquid thrombin solution. The flowable hemostatic colloid has a collagen concentration in the range of approximately 10%-20% weight/volume (+/−5%). The collagen particles may comprise collagen microfibrils such as those sold under the trademark AVITENE. The collagen particles have a bulk density in a range of approximately 1.5 lbs/ft³ to about 3.5 lbs/ft³. When formed, the flowable hemostatic colloid has a viscosity of less than 3300 centipoise, between approximately 1000 centipoise and approximately 3000 centipoise (+/−100 centipoise). The flowable hemostatic colloid also has a pH in a range of approximately 4.0 to 6.0, with unexpected hemostatic results with a flowable hemostatic colloid having a pH of 4.5-5.5, ideally at approximately 5.0+/−0.1. The liquid thrombin solution is formed by mixing thrombin with saline. The liquid thrombin solution is then combined with the collagen particles and mixed until a visibly homogeneous solution is formed resulting in the desired flowable hemostatic colloid of the present invention.

The hemostatic colloid of the invention may increase hemostatic efficacy by at least three mechanisms: physical, chemical, and physiological; each of which are discussed below at greater length. In particular, the hemostatic colloid may activate the blood-coagulation factors to boost the level and formation of thrombin, and the material may absorb fluid from the blood and further expand. Application of the hemostatic colloid may increase the viscosity of blood, blood flow speed may be reduced, and the colloid may clog the opening of the blood vessel through which bleeding is taking place. Because the hemostatic material may activate the blood-coagulation factors and boost the formation of thrombin, it may notably be effective for patients with blood-coagulation obstructions or defects.

The hemostatic colloid can be used both for a broad range of uses, including clinical and for first aid. It can advantageously and easily be use in hostile environments where a simple and effective means for stopping the flow of blood or body fluids is desired (e.g., battleground situations). The hemostatic colloid may be soluble and may be used on wound surfaces under pressure. The material can be provided free of any medications, if desired, or may contain desired medications for particular purposes.

The hemostatic colloid may be suitable for use in both surgical applications as well as for use in field treatment of traumatic injuries. For example, the material may be suitable for use in vascular surgery, where bleeding can be particularly problematic. The hemostatic material may be suitable for use in cardiac surgery, where multiple vascular anastomoses and cannulation sites, complicated by coagulopathy induced by extracorporeal bypass, can result in bleeding that can only be controlled by topical hemostats. The hemostatic material may be suitable to produce rapid and effective hemostasis during spinal surgery, where control of osseous, epidural, and/or subdural bleeding or bleeding from the spinal cord is not amenable to sutures or cautery. In such instances, the hemostatic material can minimize the potential for injury to nerve roots and reduce the procedure time. In another example, the hemostatic material may also be suitable for use in liver surgery, in live donor liver transplant procedures, or in the removal of cancerous tumors; where there is a substantial risk of massive bleeding. The material may be suitable for use as an effective hemostatic material, which can significantly enhance patient outcome in such procedures. Even in situations where massive bleeding is not a problem, the hemostatic colloid may be suitable for use to achieve hemostasis. For example, the material may be used in dental procedures, such as tooth extractions; for abrasions; burns; sports related injuries, and the like. The material may also be suitable for use in neurosurgery, where oozing wounds are common and are difficult to treat.

4. Formation of a Flowable Hemostatic Collagen Colloid

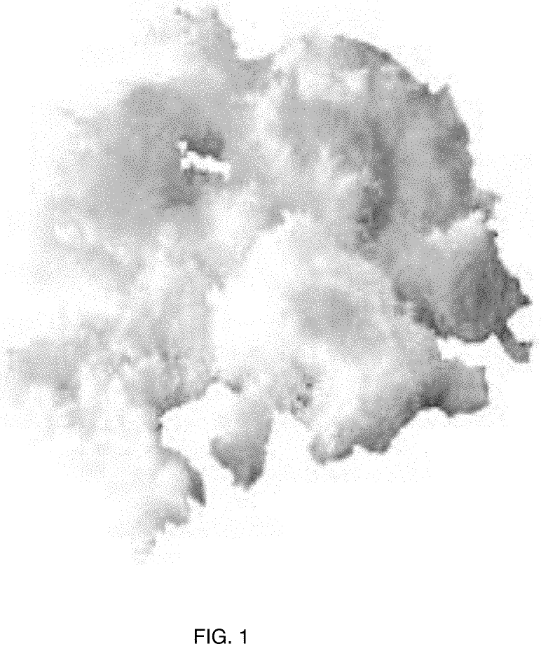

In order to form a flowable hemostatic collagen colloid from the aforementioned dried (solid) hemostatic material comprising uncrosslinked collagen as shown in FIG. 1, the solid hemostatic material is combined with a liquid and mixed until a homogenous colloid is formed. The liquid may be in the form of a thrombin solution containing thrombin and sterile water or saline. The quantity of liquid to the amount of solid hemostatic material is such that the absorption properties are not completely depleted while allowing a flowable hemostatic colloid to be formed that can be dispersed as through a syringe.

In order to form the collagen/thrombin hemostatic colloid of the present invention, a process of mixing collagen in solid crystalline, powder or granular form with a saline/thrombin solution is provided. The method for forming a collagen/thrombin hemostatic colloid comprises mixing a liquid thrombin solution with collagen in solid form. This procedure is carried out at the time when the hemostatic colloid is needed, such as during a surgical procedure when the control of bleeding tissue is desired. In order to form the collagen/thrombin hemostatic colloid, two mixing and dispensing devices in the form of two syringes 236 and 238 are interconnected with a female-to-female luer lock adapter 240. The luer lock adapter 240 is connected to and between the syringes 236 and 238 such that flow can pass only through the luer lock adapter 240 and thus between the two syringes 236 and 238. The first syringe 236 contains the liquid thrombin solution 232, such as thrombin mixed with sterile water or saline. The second syringe 238 contains the hemostatic material 234 in solid form, such as microfibrillar, crystalline, granulated or powdered collagen. By pressing the first syringe plunger 237, the liquid 232 is forced from the first syringe 236 into the second syringe 238, thereby wetting the hemostatic material 234. It is important that the syringe containing the collagen also includes pockets of air around the hemostatic material 234 between the plunger 241 and the dispensing end 242. In a first mixing step, the first syringe plunger is pressed to insert approximately half of the liquid from the first syringe into the second syringe, thereby wetting the collagen. With the air pockets present, the first and second syringes are agitated as by shaking, tipping rolling and/or rotating by hand to wet all sides of the collagen before the rest of the liquid is forced into the second syringe. Otherwise, the collagen could simply be compressed against the plunger causing the collagen to clump and not disperse throughout the thrombin solution. This causes the second syringe plunger 241 to extend. Pressing the second syringe plunger 241 toward the first syringe then forces the hemostatic material/liquid mixture from the second syringe 238 to the first syringe 236, thus causing the plunger 237 of the first syringe 236 to extend. The plunger 237 of the first syringe 236 is then pressed to force the hemostatic material/liquid mixture through the luer lock adapter 240 back into the second syringe 238. This process is repeated until a visibly homogenous mixture of the hemostatic material/liquid is formed, resulting in the formation of a hemostatic colloid. In order to view the condition of the hemostatic material/liquid until formation of the hemostatic colloid, the syringes 236 and 238 are formed from a sufficiently transparent plastic material so that their respective contents and the condition thereof can be visibly seen as mixing progresses.

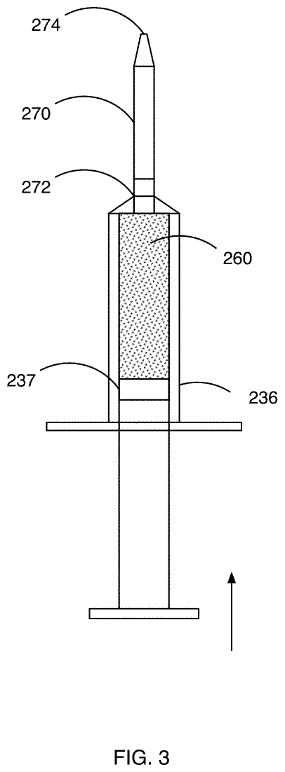

As shown in FIG. 3, the syringe 236 containing the resulting hemostatic colloid 260, which has been removed from the adapter 240 (see FIG. 3), is fitted with a dispensing adapter 270. The dispensing adapter 270 has a luer lock connector at a first end 272 and a dispensing tip 274 at a second opposite end. The first end 272 is coupled to the distal end of the syringe 236 and held together with the luer lock connection. The dispensing adapter 240 comprises a hollow tube that is configured to allow flow of the hemostatic colloid therethrough and thus to dispense the hemostatic colloid into a wound site as the plunger 237 is depressed relative to the body of the syringe as indicated by the arrow to inhibit bleeding at the wound site. Furthermore, by providing the hemostatic colloid in a syringe, the hemostatic colloid can be easily directed into a wound with the amount and location of hemostatic colloid applied easily controllable with the syringe.

By way of example, the ratio of solid hemostatic material to liquid is in a ratio of approximately 1 gram (+/−0.5 grams) of solid hemostatic material to approximately 10 mL of liquid (+/−5 mL). Thus, to form a hemostatic colloid according to the present invention, 1 gram of solid hemostatic material is mixed with 10 mL of liquid to produce a saline-based hemostatic colloid. The liquid may be comprised of a liquid thrombin solution and combined with the solid hemostatic material to form a flowable collagen/thrombin-based hemostatic colloid. For example, 1 gram of solid hemostatic material is mixed with 10 mL of thrombin solution.

The thrombin solution may be formed by combining thrombin in solid form with sterile water or sterile isotonic saline at a concentration of 1,000 to 2,000 International Units per mL. Where bleeding is profuse, as from abraded surfaces of the liver or spleen, concentrations of 1,000 International Units of thrombin per mL may be required. For general use in plastic surgery, dental extractions, skin grafting, etc. solutions containing approximately 100 International Units per mL of thrombin may be used.

In any case, an antibiotic, such as Ancef, can be added to the mixture according to a prescribed dosage. For example, 1 gram of Ancef can be mixed with 1 mL of saline and mixed with 1 gram of solid hemostatic material in 10 mL of saline or thrombin, to form a hemostatic antibiotic impregnated colloid. Such ratios of solid hemostatic material to liquid allows a flowable colloid to be formed without causing the hemostatic material to be fully saturated with the liquid. This further allows for the resulting flowable hemostatic colloid to be easily applied to a wound and allows the hemostatic colloid to further react with fluids at the wound site to assist blood coagulation.

When formed, the flowable collagen/thrombin based hemostatic colloid has the following properties:

The thrombin solution is first formed by admixing thrombin with a liquid, such as sterile water or saline. The concentration of thrombin in liquid is 1,000 to 2,000 International Units per mL, depending on the severity of bleeding and desired hemostatic properties.

The thrombin solution is then admixed with collagen in microcrystal, granular or powder form until a colloid is formed. Approximately 1+/−0.1 gram of solid hemostatic material is mixed with approximately 10+/−1 mL of thrombin solution with mixing continuing until a homogenous colloid is formed.

Surprisingly, as compared to other hemostatic agents currently in use, such as FLOSEAL or SURGIFLO by Ethicon, which have a neutral pH, the resulting flowable collagen/thrombin based hemostatic colloid has a pH of 4.5 to 5.5, although a range of approximately 4.0 to 6.0 (+/−0.5) is also advantageous. Surprisingly, the collagen based hemostatic colloid of the invention having a pH of 4.5-5.5, and ideally at a pH of approximately 5.0+/−0.1, has shown an unexpected and surprisingly improved hemostasis properties when compared to other hemostatic agents, such as FLOSEAL or SURGIFLO. For example, mixing 1 gram of a microfibrillar collagen hemostat with 10 mL thrombin/saline solution according to the invention produces a hemostatic colloid having a pH of 5.0.

That being said, it is also possible to adjust the pH of the collagen based hemostatic colloid if desired by adding, for example, sodium bicarbonate. For example, mixing 1 gram microfibrillar collagen hemostat with 8 mL thrombin and 2 mL sodium bicarbonate (1 meq/ml) has a pH of 7.1. Mixing 1 gram microfibrillar collagen hemostat with 9 ML thrombin and 1 ML sodium bicarbonate (1 meq/ml) has a pH of 6.5.

The viscosity of the flowable collagen/thrombin based hemostatic colloid is less than 3300 centipoise (that of SURGIFLO by Ethicon) and ideally in a range from approximately 1000 centipoise to approximately 3000 centipoise (+/−100 centipoise) absolute viscosity (e.g., between the viscosity of hand cream and honey at 70 degrees Fahrenheit) so as to be have a high enough viscosity to stay in place when dispensed by to still remain flowable so as to be deliverable through a syringe or other dispensing device having a relatively small orifice to control the amount and location of hemostatic colloid being dispensed.

The homogenous colloid is then administered, as through a syringe to the wound site. When compared to current hemostatic products on the market, the flowable collagen/thrombin based hemostatic colloid of the invention has significantly improved hemostatic properties. Baxter International, Inc. claims its FLOSEAL hemostatic agent provides a 2 minute median time to hemostasis. Ethicon claims that its SURGIFLO hemostatic agent provides a median time to hemostasis in approximately 1 minute. Surprisingly, the flowable collagen/thrombin based hemostatic colloid of the invention provides a median time to effective hemostasis of well under 1 minute, with median times between approximately 30 to 50 seconds (+/−1 second). Improvements in just a few seconds of time to hemostasis reduces the need for transfusions, reduces the possibility of bleeding-related complications, improves clinical outcomes and lowers hospital costs. For example, use of the flowable collagen/thrombin based hemostatic colloid of the invention can shorten surgery length by more rapidly controlling bleeding and reducing the need for surgical revisions. This can also result in fewer intensive care unit days.

The hemostatic colloid of the various embodiments exhibits good adherence to wounds such that an adhesive or other material to enhance the adhesive properties of the hemostatic colloid is generally not necessary.

5. Use of Additional Hemostatic Agents

Other suitable hemostatic agents that can be employed in various embodiments may include, but are not limited to, clotting factor concentrates, recombinant Factor Vila, alphanate FVIII concentrate, bioclate FVIII concentrate, monoclate-P FVIII concentrate, haemate P FVIII, von Willebrand factor concentrate, helixate FVIII concentrate, hemophil-M FVIII concentrate, humate-P FVIII concentrate, hyate-C®. Porcine FVIII concentrate, koate HP FVIII concentrate, kogenate FVIII concentrate, recombinate FVIII concentrate, mononine FIX concentrate, and fibrogammin P FXIII concentrate. Such hemostatic agents can be applied to the hemostatic material of this invention in any suitable form (e.g., as a powder, as a liquid, in a pure form, in a suitable excipient, on a suitable support or carrier, or the like).

A single hemostatic agent or combination of hemostatic agents can be employed. Loading levels for the hemostatic agent on the hemostatic material can vary, depending upon, for example, the nature of the selected material and hemostatic agent, the form of the material, and the nature of the wound to be treated. A weight ratio of additional hemostatic agent to hemostatic material may be from about 0.05:1 or lower to about 2:1 or higher. A weight ratio from about 0.06:1, 0.07:1, 0.08:1, 0.09:1, 0.10:1, 0.15:1, 0.20:1, 0.25:1, 0.30:1, 0.35, 0.40:1, 0.45:1, 0.50:1, 0.55:1, 0.60:1, 0.65:1, 0.70:1, 0.75:1, 0.80:1, 0.85:1, 0.90:1, or 0.95:1 to about 1:1, 1.1:1, 1.2:1, 1.3:1, 1.4:1, or 1.5:1 may also be employed, although higher or lower ratios can be used for certain embodiments.

6. Use of Auxiliary Substances in Preparing Hemostatic Materials

In certain embodiments, other materials may be used in conjunction with the hemostatic material including fibrinogen and hydrogels. Other auxiliary substances can also be employed, as will be appreciated by one skilled in the art.

7. Multifunctional Hemostatic Materials

In addition to effectively delivering a hemostatic agent to a wound, in some embodiments, the hemostatic materials can deliver other substances as well. In a particular embodiment, such substances may include medicaments, antibiotics, pharmaceutical compositions, therapeutic agents, and/or other substances producing a physiological effect. The substances can be deposited on the hemostatic material by any suitable method known in the art for depositing a material onto another material or incorporating an agent into a material.

In some embodiments, any suitable medicament, pharmaceutical composition, therapeutic agent, antibiotic or other desirable substance can be incorporated into the hemostatic material comprising the described collagen. The medicaments may include, but are not limited to, anti-inflammatory agents, anti-infective agents, antibiotics, anesthetics, immunosuppressive agents and chemotherapy agents.

Other substances which can be used in various embodiments can include, or be derived from, traditional medicaments, agents, and remedies that have known antiseptic, wound healing, and pain relieving properties.

Other substances that can be incorporated into the hemostatic material of various embodiments may include various pharmacological agents, excipients, and other substances well known in the art of pharmaceutical formulations. Other pharmacological agents may include, but are not limited to, antiplatelet agents, anticoagulants, ACE inhibitors, and cytotoxic agents.

Having described these aspects of the invention, it is understood that the invention provides a new kind of hemostatic materials and it can be made in the industry simply and economically. It is also understood that the invention defined by the appended claims is not to be limited by particular details set forth in the above description, as many apparent variations thereof are possible without departing from the spirit or scope thereof.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

What is claimed:

1. A method of preparing a hemostatic colloid, comprising:
admixing a plurality of collagen particles with a hemostatic agent in the form of a liquid thrombin solution; and
mixing the plurality of collagen particles with the liquid thrombin solution until a flowable hemostatic colloid is formed, wherein the collagen particles have a bulk density sufficient to form a suspension in the liquid thrombin solution.

2. The method of claim 1, wherein the flowable hemostatic colloid has a collagen concentration in the range of approximately 10%-20% weight/volume.

3. The method of claim 1, wherein the collagen particles comprise collagen microfibrils.

4. The method of claim 1, wherein the collagen particles have a bulk density in a range of approximately 1.5 lbs/ft³ to about 3.5 lbs/ft³.

5. The method of claim 1, wherein the collagen particles are in the form of a powder or in a crystalline or granular form.

6. The method of claim 1, wherein the flowable hemostatic colloid has a viscosity of less than 3300 centipoise.

7. The method of claim 6, wherein the flowable hemostatic colloid has an absolute viscosity of between approximately 1000 centipoise and approximately 3000 centipoise.

8. The method of claim 1, wherein the flowable hemostatic colloid has a pH in a range of approximately 4.0 to 6.0.

9. The method of claim 8, wherein the flowable hemostatic colloid has a pH of 4.5-5.5.

10. The method of claim 9, wherein the flowable hemostatic colloid has a pH of approximately 5.0+/−0.1.

11. The method of claim 1, further comprising forming the liquid thrombin solution by mixing thrombin with saline.

12. The method of claim 1, wherein the mixing comprises mixing approximately 1 gram of the collagen particles with approximately 10 mL of the liquid thrombin solution.

13. The method of claim 8, further comprising increasing the pH of the flowable hemostatic colloid by adding sodium bicarbonate to either the collagen particles or the liquid thrombin solution prior to mixing.

14. The method of claim 13, further comprising mixing approximately 1 gram of collagen particles with approximately 8 mL liquid thrombin solution and approximately 2 mL sodium bicarbonate to form the flowable hemostatic colloid with a pH of 7.1.

15. The method of claim 13, further comprising mixing approximately 1 gram collagen particles with approximately 9 ML liquid thrombin solution and approximately 1 mL sodium bicarbonate to form the flowable hemostatic colloid with a pH of approximately 6.5.

16. The method of claim 1, further comprising connecting a first syringe containing the liquid thrombin solution to a second syringe containing the collagen particles and air, dispensing a portion of the liquid thrombin solution from first syringe into the second syringe to wet the collagen particles and agitating the first and second syringes to prevent clumping of the collagen particles.

17. The method of claim 16, further comprising dispensing a remainder of the thrombin solution from the first syringe into the second syringe to form a collagen/thrombin mixture and then passing the collagen/thrombin mixture back and for the between the first and second syringes until the flowable hemostatic colloid is formed.

18. A flowable hemostatic colloid, comprising:
a plurality of uncrosslinked collagen particles homogenously mixed with a hemostatic agent in the form of a liquid thrombin solution to form a flowable hemostatic colloid;
wherein the collagen particles have a bulk density sufficient to form a suspension in the liquid thrombin solution and wherein the flowable hemostatic colloid has a collagen concentration in the range of approximately 10%-20% weight/volume.

19. The flowable hemostatic colloid of claim 18, wherein the collagen particles comprise collagen microfibrils.

20. The flowable hemostatic colloid of claim 18, wherein the collagen particles have a bulk density in a range of approximately 1.5 lbs/ft³ to about 3.5 lbs/ft³.

21. The flowable hemostatic colloid of claim 18, wherein the collagen particles are in the form of a powder or in a crystalline or granular form.

22. The flowable hemostatic colloid of claim 18, wherein the flowable hemostatic colloid has an absolute viscosity of between approximately 1000 centipoise and approximately 3000 centipoise.

23. The flowable hemostatic colloid of claim 18, wherein the flowable hemostatic colloid has a pH in a range of approximately 4.0 to 6.0.

24. The flowable hemostatic colloid of claim 23, wherein the flowable hemostatic colloid has a pH of 4.5-5.5.

25. The flowable hemostatic colloid of claim 24, wherein the flowable hemostatic colloid has a pH of approximately 5.0+/−0.1.

26. The flowable hemostatic colloid of claim 18, wherein the liquid thrombin solution comprises a mixture of thrombin and saline.

27. The flowable hemostatic colloid of claim 18, wherein the flowable hemostatic colloid comprises approximately 1 gram of the collagen particles and approximately 10 mL of the liquid thrombin solution.

28. The flowable hemostatic colloid of claim 18, further comprising sodium bicarbonate.

29. The flowable hemostatic colloid of claim 28, wherein flowable hemostatic colloid has a pH of approximately 6.5.
